(19) United States
(12) Patent Application Publication
(10) Pub. No.: US 2023/0136482 A1
(43) Pub. Date: May 4, 2023

(54) NUTRITIONAL COMPOSITION FOR GLUCOSE SUPPORT

(71) Applicant: Kate Farms, Inc., Santa Barbara, CA (US)

(72) Inventors: Maia Vernacchia, Santa Barbara, CA (US); Vanessa Millovich, Laguna Niguel, CA (US); Susan Ray, Temecula, CA (US); Melissa Bailey, Philadelphia, PA (US); Erin Lamb, Santa Barbara, CA (US)

(21) Appl. No.: 17/453,056

(22) Filed: Nov. 1, 2021

(51) Int. Cl. A23L 33/185; A61K 36/48; A23L 33/18; A23L 33/11; A23L 33/12; A23L 33/125; A23L 33/21; A23L 33/175

(52) U.S. Cl. CPC A23L 33/185; A61K 36/48; A23L 33/18; A23L 33/11; A23L 33/12; A23L 33/125; A23L 33/21; A23L 33/175; A23V 2002/00

(57) ABSTRACT

A nutritional composition for enteral feeding provides sole-source or supplemental nutrition via a nutrition shake for glycemic control that contains lower sugar and carbohydrates while being entirely plant-based and suitable for vegan, lactose-free, dairy-free, and gluten-free diets. The composition comprises pea protein, a balanced fat blend (e.g., sunflower, flaxseed, and medium chain triglycerides), an easy to digest carbohydrate blend (e.g., agave syrup, brown rice syrup solids, pea starch), with agave inulin fiber, and micronutrients. The nutritional composition has a low glycemic index while excluding artificial sweeteners, artificial flavors, corn syrup, sugar alcohols, lactose or milk protein, and common allergens. The composition includes a combination of ingredients specific to glycemic control, including plant-based omega-3 for cardiovascular health, phytonutrients such as quercetin and chlorogenic acid, and arginine to support nutritional needs of individuals with wounds. Prebiotic soluble fiber (e.g., agave inulin) is included to support the gut microbiome.

### BACKGROUND OF THE INVENTION

Diabetes Mellitus (DM) consists of abnormal carbohydrate metabolism related to absolute or relative deficiency of insulin or insulin action. Type 1 Diabetes Mellitus (T1DM) is an autoimmune process that destroys the insulin producing beta cells of the pancreas which results in little to no endogenous insulin production, whereas Type 2 Diabetes Mellitus (T2DM) is generally characterized by insulin resistance. Depending on type and etiology of DM, modes of treatment may include insulin, oral antihyperglycemic medications, non-insulin injectables, and/or diet and lifestyle interventions.

At the same time, liquid nutrition supplements are well-known and have been readily available in stores and otherwise distributed for medical purposes for many years. These compositions come in various flavors and can be consumed in addition to a regular diet, as a replacement for meals in a day, or in lieu of other food altogether. Initially, they were developed mostly for use with medical supervision by the elderly, patients recovering from illness, and others who need help putting on or retaining weight or otherwise meeting their nutritional requirements, but more have recently been marketed for use by healthy adults.

In the medical field, these compositions are used to treat and manage patients with a variety of ailments that have difficulty digesting foods and have decreased nutrient intake caused by, for example: gastroesophageal reflux, or rumination; malabsorption due to disorders such as short bowel syndrome, jejunostomy, EOE (eosinophilic esophagitis), celiac disease, cystic fibrosis, Crohn's disease or disaccharidase deficiency; endocrine dysfunction resulting from or associated with conditions such as DM and proteinuria; increased energy requirements of diseases such as bronchopulmonary dysplasia, cystic fibrosis, or hyperthyroidism; and increased energy needs due to trauma, severe burns, post-operative recovery, nutritional treatment before, during and after cancer treatment, and during times of metabolic stress as seen with neurological disorders such as cerebral palsy and amyotrophic lateral sclerosis (ALS); metabolic syndrome that presents with insulin resistance; non-alcoholic fatty liver disease (NAFLD) (e.g., non-alcoholic steatohepatitis (NASH)) which involves recommendations to limit simple sugars and control fats; illnesses such as asthma, lupus, rheumatoid arthritis, Crohn's disease, or ulcerative colitis, which are often treated with corticosteroids that reduce inflammation but also have the effect of increasing insulin resistance and increasing blood glucose levels, possibly causing steroid induced diabetes; and organ transplantation, which can involve post-operative stress induced hyperglycemia related to immunosuppressive drugs.

In addition, food allergies are increasingly common. Almost 8% of the U.S. children have some type of food allergy, and data suggest that at least 10>8% (>26 million) of U.S. adults are food allergic. Common allergens consist of milk (including whey, casein, and lactose), wheat, soybeans, eggs, peanuts, tree nuts, fish, shellfish, corn, and sesame. As nutrients are necessary for proper growth and development, removing them from a regular diet leads to malnutrition issues. In particular, removal of milks and/or milk proteins, eggs, and soybeans from diet of children can often have adverse consequences. Currently, products in the markets utilize corn sweeteners, corn starches, synthetic corn derivatives and corn oils, many of which are produced from genetically modified sources. In addition, these products utilize the hydrolyzed protein of milk, whey or casein making their products unavailable to those patients with galactosemia, who are lactose intolerant or have a milk allergy.

### SUMMARY OF THE INVENTION

In the light of above-mentioned problems, there is a need for a well designed formulation offered as a medical nutrition product or product line that addresses the issues faced by people and patients who want to rely upon a plant-based, vegan formula, in which the formula is free of common allergens and synthetic ingredients. Further, there is a need for a plant-based composition comprising plant-based protein, a beneficial blend of healthy fats, a blend of key fibers, and phytonutrients, and are suitable for enteral feeding, including both oral and tube administration.

Additionally, there is a need for a plant-based nutrition formula that is specifically intended for patients with diabetes, hyperglycemia, glucose intolerance, or glycemic control issues.

Macronutrients of food can affect blood glucose in a variety of ways. Carbohydrate is the main food source that increases blood glucose. However, depending on the type of carbohydrate, the impact may be quite variable.

In general, a dietary plan for glycemic control aims to keep blood glucose levels within a target range for a target amount of time, or “time in range” (TIR), consistently (e.g., having a high TIR) and with minimal fluctuation, an ideal known as “flat, narrow, in-range” (FNIR). Dietary plans can also be based on different foods' Glycemic Index (GI), which is a value system based on how specific foods impact blood glucose levels. Lower glycemic index diets have been demonstrated to lead to statistically significant improvements in A1c and fasting glucose compared to a high glycemic index diet.

The American Diabetes Association (ADA) does not recommend any specific eating or dietary pattern and instead recommends that meal planning should be patient centered and individualized. Individualized dietary plans can be formulated and/or adjusted based on whether they result in blood glucose levels for the individual in question that are FNIR. The ADA recommends all providers refer persons with diabetes to a Registered Dietitian Nutritionist (RDN), who typically also become credentialed as Certified Diabetes Care and Education Specialists (CDCES) when working with Diabetes patients, for individualized Medical Nutrition Therapy (MNT) to develop a plan tailored for their lifestyle. RDN/MNT/CDCES intervention is associated with a decrease in Hemoglobin A1c of 1.0-1.9% in T1DM, and a 0.3-2% decrease in A1c in T2DM.

Diabetes Specific Formulas (DSF) are formulas that are designed specifically to meet the metabolic goals of those individuals with diabetes to optimize glycemic control. DSF's were found to be more effective at lowering postprandial blood glucose levels compared to standard formula. In these studies, DSFs also improved glycemic control, lowered insulin requirements, and were associated with better clinical outcomes including a reduced risk of acquired infections and pressure ulcers.

A common complication of diabetes is gastroparesis. Gastroparesis is delayed gastric emptying; in the setting of diabetes, long term hyperglycemia is the cause of this complication. Nutritional therapy for gastroparesis includes using liquids which are generally better tolerated due to liquids emptying by gravity and not requiring antral contraction to leave the stomach. Therefore, even liquids that contain fat are generally well tolerated. For oral diets including solid foods, it is generally recommended to reduce fiber and fat, which can delay gastric emptying. Low GI formulas may be helpful for improving glycemic control which may help with alleviating symptoms of diabetic gastroparesis. Hyperglycemia at 140 mg/dL or higher, in the absence of neuropathy, disrupts normal antral motor complexes in healthy humans. Gastric emptying improves during euglycemia. Therefore, although gastroparesis is a consequence of long-term hyperglycemia, symptom management may improve with present glycemic control.

Plant-based diets, which emphasize whole foods and less processed/animal-based foods, are gaining popularity in diabetes care due to their potential beneficial components such as dietary fiber, phytonutrients, healthier fats, and little to no cholesterol. A 2014 meta-analysis found significantly lower A1c of nearly 0.4% for those individuals on a vegetarian or vegan diet vs a traditional non-vegetarian diet. Vegetarian groups had lower energy intake, protein, total fat, and higher intakes of carbohydrate and fiber. A reduction in energy intake is typically associated with weight loss, which is known to improve glycemic control. However, there are other studies that have seen improved glycemic control with vegetarian diets in the absence of weight loss. On the other hand, processed and unprocessed meat intake has been associated with a 30% increased risk in developing T2DM.

Dietary fiber can reduce the risk of developing T2DM and affect glucose metabolism by several mechanisms including lowering the glycemic index of carbohydrates, slow absorption in the small intestine, and stimulating secretion of glucagon-like peptide 1 (GLP-1). Dietary fiber has numerous health benefits and can affect the gut microbiome, immunity, laxation, cardiometabolic risk factors, and more.

Plants contain many phytonutrients which have shown beneficial effects on diabetes prevention, glucose metabolism and blood sugar management, inflammation, the gut microbiome, and slowing the progression of complications such as retinopathy, neuropathy, and nephropathy. Phytonutrients such as bitter melon, green tea, and cinnamon cassia (to name a few examples) also have beneficial effects on cardiovascular health or cardiovascular disease (CVD), providing antioxidant functions, reducing platelet aggregation, and supporting blood vessel health particularly in diabetics. Phytonutrients such as quercetin and chlorogenic acid show promise as adjuncts in diabetes care and management. Other beneficial phytonutrients include berberine, citrus bioflavonoids, amla (Indian gooseberry), and fenugreek.

Quercetin is a plant flavanol and flavonoid that is found in various fruits, vegetables, leaves, seeds, and grains, including kale. Improvements in insulin resistance, glucose metabolism, islet beta cell protection, as well as protective effects on diabetic complications such as retinopathy or neuropathy have been demonstrated with the use of quercetin in animal models. Additionally, long term hyperglycemia is one cause of increased oxidative stress and advanced glycation end product formation, associated with inflammation and progression of diabetes complications. In vitro studies have shown that quercetin effectively protected against diabetes induced retinopathy by reducing oxidative stress mediated damage. A human pilot study of quercetin, chlorogenic acid, and myricetin supplementation demonstrated similar results when compared to the use of metformin. These results suggest a beneficial effect of quercetin in diabetes care.

Chlorogenic acid occurs in various plants, including fruits, vegetables, green tea, and coffee. Supplementation of chlorogenic acid has demonstrated a reduced production of ROS, thereby protecting beta cells as well as enhancing antioxidant enzymes activity such as superoxide dismutase, in rodent models. A human pilot study utilizing a chlorogenic acid supplement 3 times per day in patients with impaired glucose tolerance found significant decreases in fasting plasma glucose compared to the control.

As nutrients are required for proper growth and development and to support the health and maintain the normal structure of bone, muscle and key physiological processes, removing nutrients from a regular diet as a result of food allergies can lead to malnutrition issues. Individuals with diagnosed food allergies benefit from well-designed medical nutrition formulas, especially in circumstances where glucose support associated with abnormal glucose tolerance is required.

Based on this understanding, the present invention relates to nutritional compositions for use in enteral feeding, including tube and oral feeding, that are allergen-free, plant-based and preferably entirely plant-based and vegan, organic, free of ingredients from genetically modified organisms (GMOs), and free of synthetic ingredients. These nutritional compositions should comprise a protein source (e.g., pea protein, hydrolyzed pea protein, fava protein, rapeseed protein, or sacha inchi protein), phytochemical extracts, fatty acids, organic ingredients free of common allergens and corn, and prebiotic fibers. In addition, the compositions should avoid ingredients that have been previously used to inexpensively meet macro or micronutrient levels (or other ingredients for flavor, color, viscosity, etc). In addition, the nutritional composition is preferably calorically dense so that nutrition needs can be met with less volume. Generally, the caloric density is between 1 kcal/mL and 2 kcal/mL. In one embodiment, the caloric density is less than 1.5 kcal/mL and is preferably about 1.2 kcal/mL.

The presently disclosed nutritional composition for enteral feeding provides sole-source or supplemental nutrition via a specialized formula or nutrition shake for glycemic control that contains lower sugar and carbohydrates in addition to being entirely plant-based and suitable for plant-based, vegan, lactose-free, dairy-free, and gluten-free diets. The nutritional composition helps manage blood sugar, having a low glycemic index while excluding artificial sweeteners, flavors, corn syrup, or sugar alcohols, and does not use maltodextrin as a primary carbohydrate source, instead using natural organic flavors (e.g., vanilla, chocolate, coffee), agave syrup, and brown rice syrup solids. Moreover, the nutritional composition provides the benefits of a plant-based diet but does not contain any lactose or milk protein, improving ease of digestion. The composition includes plant-based omega-3 for cardiovascular health and phytonutrients (having antioxidant properties) in a clinically effective amount for immune support. Naturally occurring arginine is also included to support nutritional needs of individuals with wounds, for example. Naturally occurring glutamine support gastrointestinal and immune health. The composition does not contain any of the common allergens. Prebiotic soluble fiber is included to support the gut microbiome.

More particularly, the presently disclosed nutritional composition is a nutritionally complete enteral formula comprising pea protein, a balanced fat blend (e.g., sunflower, flaxseed, and medium chain triglycerides derived from coconut), an easy to digest carbohydrate blend (e.g., agave syrup, brown rice syrup solids), with inulin fiber (e.g., sourced from agave or chicory), and micronutrients. The formula is plant-based, vegan, free of major allergens (milk, wheat, soy, eggs, peanuts, tree nuts, fish, shellfish), and corn-free, non-GMO, kosher, and made with a blend of phytonutrients. The composition also has a pleasing taste and can be taken orally.

The nutritional composition is designed to promote glycemic control. Low glycemic index diets have been shown to have greater improvements in glycemic control parameters (fasting glucose and A1c), compared with high glycemic index diets. The nutritional composition has a low glycemic index, providing, for example, an 11-34% reduction in carbohydrates compared to standard (e.g., not diabetes-specific) formulas. The composition contains a blend of complex and simple carbohydrates to create a favorable blood glucose response, including organic agave syrup, which is fructose-based and helps contribute to the composition's low glycemic index. In one example, the nutritional composition has a glycemic index of 18+/−2. The nutritional composition might only have 36 grams (g) of sugar per liter. The composition can be based on a formula comprising 64 grams of plant-based protein and 20 grams of prebiotic soluble fiber per liter of the composition. The nutritional composition can also provide chromium picolinate and/or naturally occurring chromium, which may support glucose metabolism. The nutritional composition can also comprise allulose, which is a low-calorie sweetener and a monosaccharide that is present in small quantities in raisins, figs, maple syrup, jackfruit, caramel sauce, and wheat and can be synthesized from fructose. Allulose tastes like sugar but has one tenth of the calories and does not have an effect on blood glucose. The nutritional composition can also include pectin, which is a dietary fiber that becomes viscous in a low pH environment, allowing for smooth viscosity to flow through the digestive tract but also provides delayed gastric emptying, improvements in lipid profile, and improved glycemic control. Currently, the nutritional composition comprises a protein source that may include bioactive peptides, which may influence blood glucose metabolism.

The nutritional composition preferably comprises a prebiotic fiber (e.g., agave inulin), which is easy to digest, promotes the growth of beneficial bacteria in the gut such as Bifidobacteria, and reduces inflammation. Agave inulin is a soluble, prebiotic source of fiber. In a study of healthy adults, agave inulin intakes of 5.0 g and 7.5 g per day were shown to positively alter the gut microbiome. In addition, high fiber diets have been associated with improvements in glycemic control parameters (fasting glucose and A1c) compared to low fiber diets. In one embodiment, the composition includes 20 g per liter of prebiotic soluble fiber from organic agave inulin. The nutritional composition might also include resistant starch, which is a source of fermentable, prebiotic, dietary fiber that is generally well tolerated due to its large insoluble properties that allow it to ferment in the length of the gastrointestinal tract.

The nutritional composition also uses high quality, plant-based protein such as pea protein. Many medical conditions require higher protein intakes to make up for muscle loss, or enhanced tissue catabolism caused by inflammatory cytokines or elevated basal metabolic rates (e.g., infection, liver and kidney disease). After surgery, patients have elevated metabolic rates requiring both energy and protein for tissue repair. Finally, severe acute illness, burn injury, end-stage renal disease in patients on dialysis, and nursing home residents all require higher proteins needs.

Thus, in one example, the nutritional composition has 21% of its calories provided by high-quality protein such as pea protein. In fact, 100% of the protein in the nutritional composition can be from organic yellow pea. The nutritional composition might also contain tryptophan and/or sulfur-containing amino acids (SAAs).

In one embodiment, the nutritional composition includes a specially formulated enhanced mineral profile to support absorption of plant-based protein, including iron, zinc, and copper, which support protein synthesis, and/or chelated minerals.

Pea protein is high in branched chain amino acids, as well as lysine and arginine, which is helpful in promoting wound healing of diabetic ulcers. In addition, pea protein (e.g., pea protein isolate and/or pea protein concentrate) is unique (among plant proteins) in having a high concentration of lysine, branched chain amino acids, arginine, and glutamine/glutamic acid.

Branched-chain amino acids are essential nutrients that the body obtains from proteins found in food, especially meat, dairy products, and legumes. They include leucine, isoleucine, and valine. “Branched-chain” refers to the chemical structure of these amino acids. Branched-chain amino acids have been used for medicine, as they stimulate the building of protein in muscle and possibly reduce muscle breakdown and also seem to prevent faulty message transmission in the brain cells of people with advanced liver disease, mania, tardive dyskinesia, and other types of disease.

Arginine is a semi-essential amino acid that is vitally important for nitric oxide generation and many other essential benefits including toxin elimination, immune system maintenance and faster wound healing. Pea protein is unique in that it contains almost three times the amount of arginine versus milk proteins such as whey. High arginine plasma levels are critical for growth and development, and several studies of muscle growth in weightlifters using whey protein versus pea protein showed identical results in the anabolic effects on muscle tissue.

Moreover, the nutritional composition is designed to promote glycemic control while enhancing tolerability and digestibility. Due to its unique globular and low solubility characteristics, pea protein is considered an “intermediate” protein in digestibility, somewhere between whey (fast) and casein (slow). Pea protein also provides a high ease of digestion, with several studies showing that the true digestibility of pea protein (over 95%) is very similar to casein and eggs. Casein forms a “curd” in the stomach due to low stomach pH. Pea protein forms a similar but less strong curd that allows easier and quicker digestion and passage into the small intestine. The nutritional composition has a Protein Digestibility Corrected Amino Acid Score (PDCAAS) of 0.8-1.0, preferably 1.0. In one embodiment, the nutritional composition comprises organic intact pea protein and supplemental essential amino acids to provide a complete amino acid profile.

Preferably, the nutritional composition provides 64 g/L of plant-based protein, with a PDCAAS of 1.0, and arginine at 5.6 g/L to support wound healing.

In one embodiment, the nutritional composition contains no non-nutritive sweeteners, sugar alcohols, or aspartame.

The nutritional composition is also free of the most common food allergens (e.g., milk, wheat, soy, eggs, peanuts, tree nuts, fish, shellfish, and sesame) and corn and is gluten-free and suitable for patients with gluten allergies or Celiac disease.

Moreover, the composition comprises a polyphenol blend of phytochemical extracts and concentrates, which are high in antioxidant properties and help maintain quality and reduce oxidation throughout the shelf life of the composition. A 100 milligram (mg) dose of one polyphenol blend was shown to reduce markers of oxidative stress in healthy adult subjects.

The nutritional composition preferably comprises a blend of essential Omega-6 (e.g., sourced from high linoleic sunflower oil and/or flaxseed oil) and Omega-3 fatty acids (e.g., sourced from flaxseed oil), plus the easily digestible organic medium chain triglycerides (MCTs) derived from organic coconut oil. Omega-6 is an essential fatty acid. Omega-3 has many anti-inflammatory properties. An omega 6:3 ratio of less than 4:1 has been shown to be beneficial for reducing mortality and decreasing inflammation. MCTs are easily digested and provide an immediate energy source via a portal vein, and MCT oil helps to facilitate fat absorption. Additionally, flaxseed oil, MCT oil, and sunflower oil provide viscosity and improve the mouthfeel of the composition. In one embodiment, the nutritional composition comprises docosahexaenoic acid (DHA) sourced from algae or other plant-based sources, which is a fatty acid typically found in fatty fish, is in the class of omega-3 fatty acids, and is a primary structural component in the brain and retina. In one embodiment, the nutritional composition comprises eicosapentaenoic acid (EPA) and similar compounds such as Resolvins. In one embodiment, the nutritional composition has an omega-6:3 ratio of 3:1.

In one embodiment, the nutritional composition comprises pea starch, which increases viscosity, helping to keep the composition in solution in order to flow smoothly through a feeding pump. In one embodiment, the pea start contributes less than 4 grams of carbohydrates per 1 L of nutritional composition.

The nutritional composition might also comprise locust bean gum, which is a stabilizer that increases viscosity and also improves mouthfeel.

The nutritional composition might also comprise gum acacia, which is a natural emulsifier that also improves mouthfeel and soon may be an FDA-approved fiber source.

The nutritional composition might further comprise rosemary extract, which is an antioxidant that helps to maintain the quality of the composition and reduce oxidation throughout the composition's shelf life.

In addition, brown rice syrup solids can be added. These are carbohydrates that, at high dextrose equivalents (DE), prevent viscosity of the composition from becoming too high. For example, the nutritional composition might have brown rice syrup solids with a DE of 30.

Preferably, the nutritional composition has a ratio of hydrolyzed to intact pea protein that keeps the composition in solution and reduces settling during shelf life.

Sunflower lecithin can be added. This is an emulsifier that helps keep the composition in solution in order to flow smoothly through a feeding pump.

In one embodiment, the nutritional composition is provided in cartons, each of which contains approximately 250 mL of the composition.

In general, according to one aspect, the invention features a nutrient composition for glycemic control, the nutrient composition being in liquid, powder, bar, or pudding form, for enteral feeding, including tube and/or oral feeding. The nutrient composition comprises pea protein and/or pea protein hydrolysate, phytochemical extracts, fatty acid-containing components, organic ingredients, vitamins and minerals, and prebiotic fibers.

In embodiments, the pea protein isolate and/or pea protein hydrolysate constitutes 7.5 wt % to 8.5 wt % of the total composition and approximately 100 wt % of the total protein of the composition. The prebiotic fibers comprise organic agave inulin and possibly pea protein fiber constituting 1.5 wt % to 2.7 wt % of the total composition and approximately 100 wt % of the total dietary fiber of the composition. The organic ingredients include organic agave syrup constituting 3.2 wt % to 4.2 wt % of the total composition and approximately 75 wt % of the total sugars of the composition, which are approximately 36 g/L.

In addition to the organic agave syrup, the organic ingredients further comprise organic brown rice syrup solids constituting 2.8 wt % to 5.2 wt % of the total composition and organic pea starch constituting 0.05 wt % to 0.55 wt % of the total composition, with the organic brown rice syrup solids, organic agave syrup, and organic pea starch constituting approximately 100% of the total carbohydrates of the composition. The phytochemical extracts include at least quercetin and chlorogenic acid and constitute approximately 0.02 wt % to 0.06 wt % of the total composition. The fatty-acid containing components comprise organic sunflower lecithin constituting 0.2 wt % to 0.6 wt % of the total composition, organic flaxseed oil constituting 0.7 wt % to 1.7 wt % of the total composition, organic high linoleic sunflower oil constituting 1.9 wt % to 2.9 wt % of the total composition, and organic medium chain triglyceride (MCT) oil constituting 1.1 wt % to 2.1 wt % of the total composition. The organic ingredients further include organic locust bean gum constituting 0.01 wt % to 0.25 wt % of the total composition. The nutrient composition further comprises rosemary extract constituting 0.02 wt % to 0.04 wt % of the total composition, purified water constituting approximately 74 wt % of the total composition, and/or arginine at 5.8 g/L.

In one embodiment, the nutrient composition has a caloric density of 1.2 kcal/mL, a caloric distribution of 21% protein, 34% carbohydrates, and 45% fat, and a glycemic index of 18+/−2.

In one embodiment, the organic agave syrup and organic brown rice syrup solids constitute approximately 100 wt % of the total sugars of the composition.

In one example, the pea protein isolate and/or pea protein hydrolysate constitutes 7.5 wt % to 8.5 wt % of the total composition and approximately 100 wt % of the total protein of the composition, the prebiotic fibers comprise organic agave inulin constituting 1.5 wt % to 2.7 wt % of the total composition and approximately 65 wt % of the total dietary fiber of the composition, the total protein of the composition is 64 g per 1 L of the composition, and the total dietary fiber of the composition is 20 g per 1 L of the composition.

In general, according to another aspect, the invention features a method of preventing, reducing, and/or treating diabetes, hyperglycemia, glucose intolerance, and/or glycemic control issues. The method comprises administering a nutrient composition to a human subject in need thereof. The nutrient composition, which is in liquid form for enteral feeding, comprises pea protein and/or pea protein hydrolysate, phytochemical extracts, fatty acid-containing components, organic ingredients, and prebiotic fibers.

The above and other features of the invention including various novel details of construction and combinations of parts, and other advantages, will now be more particularly described with reference to the accompanying drawings and pointed out in the claims. It will be understood that the particular method and device embodying the invention are shown by way of illustration and not as a limitation of the invention. The principles and features of this invention may be employed in various and numerous embodiments without departing from the scope of the invention.

### DETAILED DESCRIPTION OF THE PREFERRED EMBODIMENTS

The invention now will be described more fully hereinafter with reference to the accompanying drawings, in which illustrative embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art.

As used herein, the term “and/or” includes any and all combinations of one or more of the associated listed items. Also, all conjunctions used are to be understood in the most inclusive sense possible. Thus, the word “or” should be understood as having the definition of a logical “or” rather than that of a logical “exclusive or” unless the context clearly necessitates otherwise. Further, the singular forms and the articles “a”, “an” and “the” are intended to include the plural forms as well, unless expressly stated otherwise. It will be further understood that the terms: includes, comprises, including and/or comprising, when used in this specification, specify the presence of stated features, integers, steps, operations, elements, and/or components, but do not preclude the presence or addition of one or more other features, integers, steps, operations, elements, components, and/or groups thereof. Further, it will be understood that when an element, including component or subsystem, is referred to and/or shown as being connected or coupled to another element, it can be directly connected or coupled to the other element or intervening elements may be present.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

The disclosed nutritional composition is a nutritional composition which is composed of mainly organic and plant-based ingredients. In one embodiment, the nutritional composition comprises at least one of a protein source, phytochemical extracts, one or more fatty acids, one or more non-allergic organic ingredients, and one or more prebiotic fibers. The organic ingredients are free of corn. The nutritional composition further comprises one or more vitamin and mineral blends, and purified water.

Typically, the nutritional composition further comprises organic brown rice syrup solids, organic agave syrup, and organic flavors (e.g., vanilla).

The nutritional composition is provided in liquid form for enteral feeding, including tube and/or oral feeding, and can be used as a sole source of nutrition or supplemental source of nutrition. For tube feeding, the composition can be administered via pump, gravity, or bolus. In one embodiment, the nutritional composition is provided in cartons, each of which contains approximately 250 mL of the composition.

The nutrient composition has a caloric density between 1 kcal/mL and 2 kcal/mL and preferably less than 1.5 kcal/mL. In one embodiment, the caloric density is approximately 1.2 kcal/mL.

The composition has a caloric distribution of 17% to 25% protein, 30% to 38% carbohydrates, and 41% to 49% fat. In one embodiment, the composition has a caloric distribution of 21% protein, 34% carbohydrates, and 45% fat.

The present invention also provides an easily digestible organic plant-based peptide, which is a formulation of a vegan product free from the top eight allergens and corn. Specifically, the composition contains no milk, wheat, soybeans, eggs, peanuts, tree nuts, fish and shellfish, corn, and sesame and is non-GMO (free of genetically modified organisms).

The plant-based protein source is preferably organic pea protein (e.g., from organic yellow pea (Pisum sativum), including intact pea protein and/or hydrolyzed pea protein. The concentration of the pea protein (e.g., pea protein isolate and/or pea protein hydrolysate) ranges from 6.5 wt % to 9.5 wt % of the total composition, or preferably 7.5 wt % to 8.5 wt %. The pea protein constitutes 50% to 100% of the total protein of the composition, or preferably 100% of the total protein.

Nevertheless, the composition provides a complete amino acid profile.

Pea protein is high in branched chain amino acids leucine, isoleucine, and valine in addition to lysine, arginine, and glutamine. The branched chain amino acids support protein synthesis and support muscle tissue maintenance and formation. Both the branched chain amino acids as well as arginine and glutamine have key roles in supporting wound healing such as for diabetic ulcers. Thus, the nutritional composition includes arginine to support the nutritional needs of those with wounds. The nutritional composition includes 4.6 to 6.7 g of arginine per 1 L, or preferably approximately 5.6 g/L, and 8.4 to 12.2 g of glutamine per 1 L, or preferably approximately 10.3 g/L.

The nutritional composition does not contain any lactose or milk protein.

The composition provides a Protein Digestibility Corrected Amino Acid Score (PDCAAS) ranging from 0.8 to 1.0, or preferably 1.0.

The composition also includes an organic phytonutrient blend such as a plant-based, specifically defined polyphenol blend from select organic fruits, vegetables, and herbs, which comprises one or more phytochemical extracts that are extracted without chemical solvents. In embodiments, the phytochemical extract is a blend of fruits, vegetables, herbs and spices, or the phytochemical blend is a combination of phytochemical extracts from fruits, vegetables, herbs, and spices, and organic rosemary extract. In some embodiments, the blend of phytochemical extracts is sourced from a group of fruits, vegetables, herbs, and spices, comprising one or more extracts or concentrates of broccoli sprouts, camu camu, tomato, acai, turmeric, garlic, basil, oregano, cinnamon, elderberry, chokeberry, raspberry, spinach, kale, brussels sprouts, extracts of green arabica coffee bean, green tea, onion, apple, acerola, Japanese pagoda tree (quercetin), blackcurrant, blueberry, bilberry, maqui berry concentrated juice powder, grape seed extract, or any combination thereof. In one embodiment, the phytonutrient blend includes extracts and concentrates from: organic broccoli, organic whole fruit of the coffee plant, organic green tea, organic turmeric, organic kale, organic broccoli sprout, organic acai, organic cinnamon, organic garlic, organic tomato, organic blueberry, organic carrot, organic beet, organic raspberry, organic spinach, organic tart cherry, and organic blackberry. The nutritional composition comprises a phytonutrient blend at a concentration ranging from 0 wt % to 0.4 wt %, or preferably 0.02 wt % to 0.1 wt %. Moreover, in the current embodiment, the nutritional composition comprises a phytonutrient blend of 400 mg per 1 L of the composition.

In addition, the composition further includes organic rosemary extract. The nutritional composition comprises organic rosemary extract at a concentration ranging from 0 wt % to 0.2 wt %, or preferably 0.02 wt % to 0.04 wt %. The rosemary-based extract is a natural oxidation-inhibiting product.

In general, these natural supplements effect biological changes of oxidative and nitrosative stress markers (free radicals), nitric oxide levels, and cellular metabolic activity.

The composition also preferably includes organic MCT oil. MCT is easily digestible, which acts as an immediate energy source via portal vein. The nutritional composition comprises organic medium chain triglycerides (e.g., MCT oil) at a concentration ranging from 0.5 wt % to 2.7 wt %, or preferably 1.1 wt % to 2.1 wt %. In some embodiments, the MCT is a form of saturated fatty acid from coconut with medium-chain fats between 6-12 carbons.

The composition further includes organic high linoleic sunflower oil and organic flaxseed oil in combination with the MCT oil. This provides a balanced blend of essential omega-6 fatty acids from sunflower and flax and omega-3 fatty acids such as ALA (alpha-linolenic acid) from flax, and easily digestible medium chain triglycerides (MCT) from coconut oil. In one embodiment, the omega-6 fatty acid is an essential fatty acid. Omega-3 has many anti-inflammatory properties. The MCT and long chain triglycerides (LCT) ratio ranges from 10:90 to 50:50, and the omega-6 and omega-3 ratio ranges from 2:1 to 4:1. In one embodiment, the MCT and long chain triglycerides (LCT) ratio is optimized to 30:70, and the omega-6 and omega-3 ratio is formulated to 3:1.

The composition comprises organic sunflower lecithin at a concentration ranging from 0 wt % to 0.8 wt %, or preferably 0.2 wt % to 0.6 wt %. Lecithin is a naturally occurring substance found in the membranes of living cells. Lecithin is extracted from sunflower seeds.

The composition further includes organic flaxseed oil at a concentration ranging from 0.5 wt % to 1.9 wt %, or preferably 0.7 wt % to 1.7 wt %. The organic flaxseed oil is a colorless to yellowish oil obtained from the dried, ripened seeds of the flax plant.

The composition further includes organic high linoleic sunflower oil at a concentration ranging from 1 wt % to 3.8 wt %, or preferably 1.9 wt % to 2.9 wt. The organic high linoleic sunflower oil is a variety of sunflower oil containing nearly 70% polyunsaturated linoleic acid.

The composition also preferably includes organic vanilla flavor at a concentration ranging from 0.2 wt % to 1.7 wt %, or preferably 0.8 wt % to 1.4 wt %. containing vanilla plant extract at a concentration ranging from 0.001 wt % to 0.5 wt %, or preferably 0.005 wt % to 0.1 wt %. The organic vanilla plant extract is derived from orchid seed pods of the genus Vanilla. The vanilla plant extract contains several medicinal properties, which could be useful for tackling several ailments. It is also a rich constituent of flavonoids, alkaloids, glycosides, carbohydrates and several other phytochemicals. The vanilla plant extract also contains both analgesic, antispasmodic, antioxidant and anti-inflammatory properties. In embodiments, the composition includes the organic vanilla plant extract to effect a vanilla flavoring for the composition, and/or the composition includes the organic vanilla plant extract as a base, with additional flavors added for flavor change. In one example, the composition has a plain or neutral flavor (or is unflavored) while still including the organic vanilla plant extract for its medicinal properties. In another example, the composition has an overall flavor other than vanilla (e.g., chocolate, coffee) while still including the organic vanilla plant extract for its medicinal properties and/or as a base flavor.

The nutritional composition does not include any artificial sweeteners, sugar alcohols, artificial flavors, or corn syrup, and does not use maltodextrin as a primary carbohydrate source.

The composition also includes a vitamin and mineral blend at a concentration ranging from 0.5 wt % to 1.7 wt %, or preferably 0.7 wt % to 1.5 wt %. In one embodiment, the blend may include Magnesium Citrate, Choline Bitartrate, Tricalcium Phosphate, Sodium Chloride, Potassium Chloride, Dimagnesium Phosphate, Sodium Ascorbate, Inositol, Potassium Citrate, DL-Alpha-Tocopheryl Acetate, Beta Carotene, Ferric Pyrophosphate, Vitamin A Palmitate, Sodium Molybdate, Zinc Oxide, Copper Sulfate, Manganese Sulfate, Chromium Picolinate, Sodium Selenite, Calcium Pantothenate, Niacinamide, Biotin, Pyridoxine Hydrochloride, Folic Acid, Thiamine Hydrochloride, Riboflavin, Phytonadione, Potassium Iodide, Cyanocobalamin (Vitamin B12), Cholecalciferol (Vitamin D3), sodium citrate, monosodium phosphate, disodium phosphate, monopotassium phosphate, dipotassium phosphate, chromium chloride, magnesium chloride, magnesium gluconate, and calcium carbonate.

In some embodiments, the composition includes alpha lipoic acid at a concentration ranging from 0.075 wt % to 0.9 wt %, or preferably 0.11 wt % to 0.23 wt %. The alpha lipoic acid helps with diabetic neuropathy.

In some embodiments, the composition includes sterols or stanols at a concentration ranging from 0.1 wt % to 1.5 wt %, or preferably 0.1 wt % to 0.75 wt %, which reduce serum cholesterol in individuals with high LDL cholesterol.

In some embodiments, the composition includes lutein for eye health at a concentration ranging from 0.003 wt % to 0.01 wt %, or preferably 0.003 wt % to 0.008 wt %.

The composition further comprises purified water at a concentration ranging from 66 wt % to 86 wt %, or preferably 71 wt % to 81 wt %. In the current embodiment, the concentration of purified water is 76 wt %.

The composition also includes organic locust bean gum at a concentration ranging from 0 wt % to 0.3 wt %, or preferably 0.01 wt % to 0.25 wt %.

The composition also includes prebiotic fibers (particularly organic agave inulin and possibly pea fiber) at a concentration ranging from 0.8 wt % to 3.0 wt %, or preferably 1.5 wt % to 2.7 wt %. The prebiotic fibers constitute 25% to 100% of the total dietary fiber of the composition, or preferably 80% of the total dietary fiber. In one embodiment, the organic agave inulin constitutes 1.5 wt % to 2.7 wt % of the total composition and approximately 65 wt % of the total dietary fiber of the composition. The organic agave inulin is a highly soluble dietary fiber (inulin-type fructan) that provides numerous health benefits. Inulin serves as a “fertilizer” to sustain beneficial bifidobacteria in the large intestine. In one embodiment, the nutritional composition comprises prebiotic fibers that include gum acacia, which is a natural emulsifier that also improves mouthfeel and has been shown to improve microbiome health.

Prebiotic fiber is clinically proven to be easy to digest (low gas potential) and promote the growth of good bacteria in the gut such as bifidobacteria. Further, Fecal Actinobacteria and Bifidobacterium were enriched 3- and 4-fold after ingestion of agave inulin, a long chain fructo-oligosaccharide (FOS), and desulfovibrio were depleted 40% with agave inulin. Agave inulin tended to reduce fecal 4-methyphenol and pH. A positive association between intakes of agave inulin and Bifidobacterium is briefly described herein. Total dietary fiber from agave inulin was positively associated with fecal butyrate and tended to be positively associated with bifidobacterium and was negatively correlated with desulfovibrio abundance. FOS could foster Bifidobacterium colony growth, which could also serve as food for less desirable strains of bacteria.

The nutritional composition further comprises organic agave syrup at a concentration ranging from 2 wt % to 5 wt %, or preferably 3.2 wt % to 4.2 wt %. The organic agave syrup constitutes 25% to 100% of the total sugars of the composition, or preferably approximately 75% of the total sugars. The organic agave syrup is a natural alternative to refined sugars and artificial sweeteners, extracted from the agave plant.

The nutritional composition further comprises organic brown rice syrup solids at a concentration ranging from 0 wt % to 7 wt %, or preferably 2.8 wt % to 5.2 wt %. The organic brown rice syrup solids constitute 0% to 50% of the total sugars of the composition, or preferably approximately 25% of the total sugars.

In one embodiment, the total sugars of the composition consist of approximately 36 g of sugars per 1 L of composition.

The composition also includes organic pea starch at a concentration ranging from 0 wt % to 1.0 wt %, or preferably 0.05 wt % to 0.55 wt %.

The organic agave syrup, organic brown rice syrup solids, and organic pea starch constitute 50% to 100% of the total carbohydrates of the composition, or preferably approximately 100% of the total carbohydrates.

The nutritional composition contains a blend of complex and simple carbohydrates to create a more favorable blood glucose response. For example, the organic agave syrup is fructose-based, which helps contribute to a low glycemic index of the overall product, which ranges from 10 to 55. In one embodiment, the glycemic index is 18+/−2.

More particularly, the agave syrup contains a higher level of fructose than high fructose corn syrup. An important difference is that fructose is up to twice as sweet as sucrose, and sweeter than high fructose corn syrup. Unlike table sugar or HFCS, fructose does not cause a rapid rise and subsequent large fall in blood glucose levels, which means it has a low glycemic load or glycemic index (GI). The glycemic index is a value assigned to foods based on how slowly or how quickly those foods cause increases in blood glucose levels. The blood glucose levels above normal are toxic and can cause blindness, kidney failure, increase cardiovascular risk, or peripheral vascular disease. Food components that are low on the glycemic index (GI) scale tend to release glucose slowly and steadily. Food components that are high on the glycemic index release glucose rapidly. Low GI foods tend to foster healthy weight loss, while foods high on the GI scale help with energy recovery after exercise, or to offset hypo- (or insufficient) glycemia. For an average diet, there are no health problems associated with fructose except for some individuals, who may be fructose intolerant.

Fructose was given GRAS status in 1983 and was reconfirmed in 1996 after a study of the available clinical trials on the effects of fructose consumption. Fructose has a low glycemic index, which can be used to help control blood glucose levels in concern with a total diet plan. Agave syrup is not processed in the same manner as corn syrup. Agave naturally contains inulin, a long chain fructose molecule. Fructose, sometimes known as fruit sugar, exists naturally in fruits and honey as a single unit of fructose. It exists naturally in inulin as a long chain of fructose molecules linked together, similar to the glucose chains in starch. The bonds in inulin can be broken to make free fructose. When a person eats a food containing inulin, the body's enzymes do not break down the inulin. Instead, the inulin is consumed by bacteria in the intestine. The bacteria that consume the inulin tend to be the beneficial bacteria, the same types found in yogurts and various probiotic foods. As the inulin feeds these beneficial organisms, allowing them to outcompete the detrimental intestinal organisms, inulin is labeled a prebiotic and has many health benefits. While fructose could be made by hydrolyzing or breaking the bonds of inulin, it could also be made by using an isomerase enzyme to change glucose into fructose.

The organic brown rice syrup solid (BRSS) is a natural alternative to refined sugars and artificial sweeteners. Unlike refined table sugar, which causes the body to work harder to absorb the sucrose, brown rice syrup is able to provide body with fiber, as well as 3% of a user's recommended daily intake of sodium and potassium. As the rice syrup solids are boiled and the liquid are removed, the result is a complex of simple sugars. This facilitates slower absorption of the sugars in rice syrup by the digestive system. This is a huge advantage because in the case of regular table sugar, there is rapid swell in blood sugar level with buildup of excess fat with time. The slower absorption of the sugars also allows the user or patient to maintain their energy and prevents the negative effects of sugar including fatigue, irritability and the desire for more sugar.

All these properties indicate that BRSS also have a low glycemic index. The use of the natural sweetening ability of BRSS is unique in the space of medical grade sole source nutrition. BRSS is a far superior option than maltodextrin, commonly used as a carbohydrate in comparable products. Maltodextrin is used as a thickener, filler or preservative in many processed foods. It is an artificially produced white powder that can be enzymatically derived from any starch but is commonly made from corn, rice, potato starch or wheat. Maltodextrin is also used in carbohydrate supplements that are marketed to athletes and bodybuilders as a way to boost their energy levels. However, consuming maltodextrin in large amounts has been known to be associated in certain situations with spiked blood sugar, suppresses the growth of probiotics, toxically affect several bodily organs and systems, and causes allergic reactions or side effects.

The organic flaxseed oil has a high concentration of healthy omega-3s as fatty acids associated with healthier brains and hearts, better moods, decreased inflammation, and healthier skin and hair. Flaxseed oil contains 50% to 65% omega-3 fatty acids in the form of alpha-linolenic acid (ALA). Flaxseed is the richest plant source of the omega(co)-3 fatty acid, i.e. a-linolenic acid (ALA). Flaxseed oil is low in saturated fatty acids (9%), moderate in monosaturated fatty acids (18%), and rich in polyunsaturated fatty acid (73%). Of all lipids in flaxseed oil, a-linolenic acid is the major fatty acid ranging from 39% to 5% followed by oleic, linoleic, palmitic and stearic acids, which provides an excellent omega (co)-6:omega (co)-3 fatty acid ratio of approximately 3:1. Flaxseed oil is naturally high in antioxidants like tocopherols and beta-carotene. Flaxseed oil provides basic nutrition as well as various health benefits in reducing cancer and cardiovascular diseases, lowering LDL-cholesterol and vasodilatory functions. Therefore, flaxseed oil could be considered as a functional food. Flaxseed oil is emerging as an important functional food ingredient, because of its rich contents of a-linolenic acid (ALA, omega-3 fatty acid), lignans, and fiber. Flaxseed oil, fibers and flax lignans have potential health benefits such as, in reduction of cardiovascular disease, atherosclerosis, diabetes, cancer, arthritis, osteoporosis, autoimmune and neurological disorders.

The organic high linoleic sunflower oil comprises essential omega-6, impressive fatty acid content, which includes palmitic acid, stearic acid, oleic acid, and linoleic acid. The combination of fatty acids in the body are extremely important to maintain various elements of human health, and sunflower oil could help to maintain the balance. The balance of fatty acids found in sunflower oil, including a substantial amount of linoleic acid (an omega-6 fatty acid) is optimal for patients. Finding a healthy balance between HDL or good cholesterol (omega-3s) and LDL or bad cholesterol is very important to the health of patients. Furthermore, sunflower oil contains less than 10% saturated fats, which means that it could actually reduce overall cholesterol content in the body. Omega-6 (n-6) polyunsaturated fatty acids (PUFA) in High Linoleic Sunflower Oil (e.g., arachidonic acid (AA)) and omega-3 (n-3) PUFA (e.g., eicosapentaenoic acid (EPA)) are precursors to potent lipid mediator signaling molecules, termed “eicosanoids,” which have important roles in the regulation of inflammation.

The composition typically is engineered to have an optimal combination of omega-3 and omega-6 fatty acids which are types of essential fatty acids that could not be prepared on our own, which have to obtain them from diet. Both are polyunsaturated fatty acids that differ from each other in their chemical structure. There are two critical omega-3 fatty acids, (EPA and DHA), that the patients require. Furthermore, vegetarian sources, which the present composition utilizes, contain a precursor omega-3 (alpha-linolenic acid called ALA) that the body must convert to EPA and DHA. EPA and DHA are the building blocks for hormones that control immune function, blood clotting, and cell growth as well as components of cell membranes.

In contrast, omega-6 foods containing these fatty acids are numerous in modern diets. They are found in seeds and nuts, and the oils are extracted from them. The body also constructs hormones from omega-6 fatty acids. In general, hormones derived from the two classes of essential fatty acids have opposite effects. Those from omega-6 fatty acids tend to increase inflammation (an important component of the immune response), blood clotting, and cell proliferation, while those from omega-3 fatty acids decrease those functions. Both families of hormones must be in balance to maintain optimum health, so there are some omega-6 benefits. Many nutrition experts believe that before we relied so heavily on processed foods, humans consumed omega-3 and omega-6 fatty acids in roughly equal amounts. The imbalance between omega-3 and omega-6 fatty acids may also contribute to obesity, depression, dyslexia, hyperactivity and even a tendency toward violence. Bringing the fats into proper proportion may actually relieve those conditions. The composition of the present invention has a unique 3:1 and 4:1 ratio of omega 6:omega 3, which facilitates balance of these fatty acids and contributes to the anti-inflammatory effects of them.

The agave inulin is a highly soluble dietary fiber (inulin-type fructan) that provides numerous health benefits. Inulin serves as a “fertilizer” to sustain beneficial bifidobacteria in the large intestine. Inulin is clinically proven to be easy to digest and promotes the growth of good bacteria in the gut such as bifidobacteria. In studies, fecal actinobacteria and bifidobacterium were enriched 3- and 4-fold after ingestion of agave inulin, a long chain fructo-oligosaccharide (FOS), and desulfovibrio were depleted 40% with agave inulin compared with the control. Agave inulin tended to reduce fecal 4-methyphenol and pH. Total dietary fiber from agave inulin has been positively associated with fecal butyrate and tended to be positively associated with Bifidobacterium and was negatively correlated with desulfovibrio abundance.

The MCT (Medium-Chain Triglycerides) oil is quickly and easily digested and provides a relatively large amount of energy to the patient. It is associated with stimulation of the metabolism and has been identified as a potential weight loss aid. If MCT oil is consumed to increase weight gain, a substantial amount of protein would aid in weight gain. Medium-chain triglycerides are easily absorbed by the gastrointestinal tract and converted into energy by the liver. They are often digested well even by those who cannot digest normal, long-chain fatty acids. MCT is comprised of primarily caprylic and capric fatty acids. MCT helps to treat diseases such cystic fibrosis, and fat malabsorption. It has also been used to add calories to infant and certain other formulas. Unlike other fats, MCT oil benefits because it does not go through the lymphatic system, it is transported directly to the liver where it is metabolized so it releases energy like a carbohydrate and creates ketones.

The organic sunflower lecithin is good for heart health. It helps to prevent and reverse damage from coronary disease. Lecithin is essentially an emulsifier of fat which could be used by the body to discard the excess fat from the bloodstream. These excess fats include cholesterol and triglycerides meaning that it will help you to reduce and control the levels of cholesterol. High concentration of linoleic acid present in the sunflower lecithin helps to perform the above process. In addition, olive oil has similar levels of linoleic acid. Phospholipids also plays a vital essential role for liver health and prevents excessive accumulation of fat.

Another advantage of using sunflower lecithin is the extraction process. Sunflower lecithin gets extracted without the need for potentially damaging chemical solvents such as acetone and hexane. Sunflower lecithin is produced through a cold press system, as like deriving oil from olives and similar products. Sunflower lecithin is abundant with essential choline and fatty acids, such as phosphatidylinositol.

The organic vanilla plant extract soothes inflammation throughout the body. This is particularly effective for inflammation of the liver, which occurs due to numerous reasons, especially for immunocompromised individuals. Vanillin could help ease arthritis, gout and other inflammatory conditions. Vanilla extract has been evaluated for hepatoprotective activity against paracetamol-induced liver damage in rats. The researchers orally administered an ethanolic extract of vanilla planifolia or a control reference solution to experimental animals with hepatotoxicity induced by paracetamol. It was observed that the plant extract was able to protect the liver against the injury induced by paracetamol in rats. This was proven by the tremendous reduction in serum enzymes alanine aminotransferase (ALT), aspartate aminotransferase (AST), alkaline phosphatase (ALP) and bilirubin. The ethanolic extract of Vanilla planifolia has hepatoprotective activity against paracetamol-induced hepatotoxicity in rats. Vanilla extract components were studied for the potential use as antioxidants for food preservation and as nutraceuticals in health supplements. The extract and pure standard compounds were screened to ascertain their antioxidant properties using b-carotene-linoleate and diphenyl picryl hydrazyl (DPPH) in vitro model systems. Interestingly, the study observed and supported the potential use of vanilla bean extracts as antioxidants.

Phytochemicals are the active ingredients that promote whole body health. The extracts do not contain any source ingredients such as, protein, fat, or carbohydrates. The blend may include extracts of green tea and green coffee but does not contain any caffeine. Blend of phytochemical extracts from fruits, vegetables, herbs, and spices represents the latest evolution in the fight against potentially-damaging free radicals. The biological effects of a natural supplement on the changes of oxidative and nitrosative stress markers, as well as cellular metabolic activity, have been clinically observed in the human body. The blend of phytochemical extract has been reported to decrease ROS, increase cellular oxygen consumption in blood and mitochondria, decrease extracellular H₂O₂, and reduce TNFa-induced inflammatory response in humans. Administration of blend of phytochemical extract resulted in statistically significant long-term inhibition of mitochondrial and cellular ROS generation by as much as 17% as well as 3.5-times inhibition in extracellular NADPH system-dependent generation of O2, and nearly complete inhibition of extracellular H₂O₂ formation. This was reflected in more than two times inhibition of ex vivo cellular inflammatory response and also increases in bioavailable NO concentration. Further, there was measured synergetic, biological effects of a natural supplement on changes in OSM and cellular metabolic activity. The unique design and activity of the plant-based natural supplement, in combination with the newly developed and extended Vitality test, demonstrates the potential of using dietary supplements to modulate OSM and also opens the door to future research into the use of natural supplements for supporting optimal health.

The potassium chloride provides benefits including relief from stroke, high blood pressure, heart and kidney disorders, and anxiety and stress. It helps in enhancing muscle strength, metabolism, water balance, electrolytic functions, and nervous system. Chloride works with other electrolytes, such as sodium and potassium, to help balance acids and bases in the body. It also helps to move fluid in and out of the cells, which is also essential for preventing dehydration.

Advantageously, the nutritional composition is entirely plant-based and free of the top eight allergens such as milk, wheat, soybeans, eggs, peanuts, tree nuts, fish and shellfish, corn, and is non-GMO (free of non-genetically modified organisms). In addition, it contains organic ingredients, optimal fatty acid ratios, oxidation fighting ingredients, anti-inflammatory properties and beneficial fiber. Alternative approaches to the problem include formation of a blended food product.

In the current embodiment, the nutritional composition has: 1200 kcal per 1 L of the composition, with a caloric density of 1200 kcal/L; a caloric distribution of 21% protein, 34% carbohydrates, and 45% fat; 64 g of protein per 1 L of the composition from a plant-based protein source, namely organic pea protein; 104 g of carbohydrates per 1 L of the composition from organic brown rice syrup solids, organic agave syrup, and organic pea starch; 60 g of fat per 1 L of the composition from organic high linoleic sunflower oil, organic MCT oil, and organic flaxseed oil; 20 g of dietary fiber per 1 L of the composition from organic agave inulin; 36 g of sugar per 1 L of the composition from organic agave syrup; an MCT:LCT ratio of 30:70; an osmolality (mOsm/kg water) of 340; a renal solute load of 497 mOsm/L; an omega-6:3 ratio of 3:1; a free water percentage of 74%; 400 mg of phytonutrients per 1 L of the composition; a nonprotein calorie to nitrogen ratio of 92:1; a glycemic index of 18+/−2.

While this invention has been particularly shown and described with references to preferred embodiments thereof, it will be understood by those skilled in the art that various changes in form and details may be made therein without departing from the scope of the invention encompassed by the appended claims.

What is claimed is:

1. A nutrient composition for glycemic control, the nutrient composition being for enteral feeding and comprising:
pea protein isolate and/or pea protein hydrolysate;
phytochemical extracts;
fatty acid-containing components;
organic ingredients; and
prebiotic fibers.

2. The nutrient composition of claim 1, wherein the pea protein and/or pea protein hydrolysate constitutes 7.5 wt % to 8.5 wt % of the total composition.

3. The nutrient composition of claim 2, wherein the pea protein isolate and/or pea protein hydrolysate constitutes approximately 100 wt % of the total protein of the composition.

4. The nutrient composition of claim 1, wherein the prebiotic fibers comprise organic agave inulin constituting 1.5 wt % to 2.7 wt % of the total composition.

5. The nutrient composition of claim 4, wherein the organic agave inulin constitutes approximately 65 wt % of the total dietary fiber of the composition.

6. The nutrient composition of claim 1, wherein the organic ingredients include organic agave syrup constituting 3.2 wt % to 4.2 wt % of the total composition.

7. The nutrient composition of claim 6, wherein the organic agave syrup constitutes approximately 75 wt % of the total sugars of the composition.

8. The nutrient composition of claim 7, wherein the total sugars of the composition are approximately 36 g per 1 L of the composition.

9. The nutrient composition of claim 6, wherein the organic ingredients further comprise organic brown rice syrup solids constituting 2.8 wt % to 5.2 wt % of the total composition and organic pea starch constituting 0.05 wt % to 0.55 wt % of the total composition.

10. The nutrient composition of claim 9, wherein the organic brown rice syrup solids, organic agave syrup, and organic pea starch constitute approximately 100% of the total carbohydrates of the composition.

11. The nutrient composition of claim 1, wherein the phytochemical extracts include at least quercetin and chlorogenic acid and constitute approximately 0.02 wt % to 0.06 wt % of the total composition.

12. The nutrient composition of claim 1, wherein the fatty-acid containing components comprise organic sunflower lecithin constituting 0.2 wt % to 0.6 wt % of the total composition.

13. The nutrient composition of claim 1, wherein the fatty-acid containing components comprise organic flaxseed oil constituting 0.7 wt % to 1.7 wt % of the total composition.

14. The nutrient composition of claim 1, wherein the fatty-acid containing components comprise organic high linoleic sunflower oil constituting 1.9 wt % to 2.9 wt % of the total composition.

15. The nutrient composition of claim 1, wherein the fatty-acid containing components comprise organic medium chain triglyceride (MCT) oil constituting 1.1 wt % to 2.1 wt % of the total composition.

16. The nutrient composition of claim 1, wherein the organic ingredients include organic brown rice syrup solids constituting 2.8 wt % to 5.2 wt % of the total composition.

17. The nutrient composition of claim 1, wherein the organic ingredients include organic pea starch constituting 0.05 wt % to 0.55 wt % of the total composition.

18. The nutrient composition of claim 1, wherein the organic ingredients include organic locust bean gum constituting 0.01 wt % to 0.25 wt % of the total composition.

19. The nutrient composition of claim 1, further comprising rosemary extract constituting 0.02 wt % to 0.04 wt % of the total composition.

20. The nutrient composition of claim 1, further comprising purified water constituting approximately 76 wt % of the total composition.

21. The nutrient composition of claim 1, wherein the nutrient composition has a caloric density of 1.2 kcal/mL.

22. The nutrient composition of claim 1, wherein the nutrient composition has a caloric distribution of 21% protein, 34% carbohydrates, and 45% fat.

23. The nutrient composition of claim 1, wherein the nutrient composition has a glycemic index of 18+/−2.

24. The nutrient composition of claim 1, wherein the pea protein isolate and/or pea protein hydrolysate constitutes 7.5 wt % to 8.5 wt % of the total composition and constitutes approximately 100 wt % of the total protein of the composition, the prebiotic fibers comprise organic agave inulin constituting 1.5 wt % to 2.7 wt % of the total composition and approximately 100 wt % of the total dietary fiber of the composition, the total protein of the composition is 64 g per 1 L of the composition, and the total dietary fiber of the composition is 20 g per 1 L of the composition.

25. The nutrient composition of claim 1, wherein the nutrient composition comprises arginine at 5.6 g/L.

26. A method of preventing, reducing, and/or treating diabetes, hyperglycemia, glucose intolerance, and/or glycemic control issues, the method comprising administering to a human subject in need thereof a nutrient composition, the nutrient composition being for enteral feeding and comprising:
pea protein and/or pea protein hydrolysate;
phytochemical extracts;
fatty acid-containing components;
organic ingredients; and
prebiotic fibers.
